(19) United States
(12) Patent Application Publication
(10) Pub. No.: US 2014/0107228 A1
(43) Pub. Date: Apr. 17, 2014

(54) OILY COSMETIC

(71) Applicant: Tokiwa Coporation, Nakatsugawa-shi (JP)

(72) Inventor: Shunsuke TOGASHI, Kawaguchi-shi (JP)

(73) Assignee: Tokiwa Coporation, Nakatsugawa-shi (JP)

(21) Appl. No.: 14/010,930

(22) Filed: Aug. 27, 2013

(30) Foreign Application Priority Data
Oct. 16, 2012 (JP) 2012-229190

(51) Int. Cl. A61K 8/73; A61K 8/92

(52) U.S. Cl. CPC A61K 8/73; A61K 8/92
USPC 514/777

(57) ABSTRACT

[Problems to be solved] In an oily cosmetic product using a filling extrusion container which is used by extruding a filling from a cylindrical storage portion to a tip of ejection portion, an oily cosmetic having smooth usability, excellent makeup durability and temperature stability, and good ejection performance is provided.

[Solution] The present invention is an oily cosmetic and an oily cosmetic product. The oily cosmetic is filled and stored in a filling extrusion container, which is used by extruding a filling from a filling storage portion to the tip of ejection portion. The oily cosmetic includes (a) dextrin palmitate of 5 to 40 weight %, where the dextrin palmitate has acyl group substitution degree of less than 2.0, and (b) volatile hydrocarbon oil of 15 to 65 weight %.

### BACKGROUND OF THE INVENTION

1. Field of the Invention

The present invention relates to an oily cosmetic and an oily cosmetic product, which are used to be filled and stored in a filling extrusion container. The filling extrusion container is used by extruding a filling from a filling storage portion to a tip of ejection portion. The oily cosmetic has smooth usability, excellent makeup durability and temperature stability, and good ejection performance from the container. The oily cosmetic has also properties that there is no liquid leakage and dripping.

2. Description of the Conventional Art

Conventionally, the oily cosmetic includes, as base materials, an oil component containing solid oil and powders such as pigments, and needs functions such as good feeling in use, makeup durability, and stability. Further, in the container form to diversify, from merits of easiness of usage and portability, a filling extrusion container has been widely used. In the filling extrusion container, the cosmetic is filled and stored in the filling storage portion and then, a user uses the cosmetic by pushing the end portion or the side surface of the container to extrude the filling to the tip of ejection portion.

In these oily cosmetics, for obtaining desired usability and functionality, various oil components and powders have been selected and prepared. For example, Japanese Patent Application Laid-Open No. 2001-187715 and Japanese Patent Application Laid-Open No. 2010-116367 discuss the blending of film-forming agents and volatile oils for improving the makeup durability. Further, Japanese Patent Application Laid-Open No. 2007-261994 and Japanese Patent Application Laid-Open No. 2002-193747 discuss the stability by blending oily gelling agents, such as, organic modified clay minerals, dextrin fatty acid ester, and silicic anhydride, and high melting point waxes.

### SUMMARY OF THE INVENTION

### Problems to be solved by the Invention

However, when the film-forming agents and the volatile oils are blended in the oily cosmetic for improving the makeup durability, the powder components such as pigments easily deposit and separate, so that the stability is lacked. Further, when the oily cosmetic contains much volatile oils, liquid leakage and dripping from the container easily occur, so that there is a disadvantage that the cosmetic easily is blurred when the cosmetic is applied. Furthermore, when the oil gelling agents, such as organic modified clay minerals, dextrin fatty acid ester, and silicic anhydride, and high melting point waxes are blended for improving stability, increase of viscosity of the oily cosmetic or lowering of fluidity with the solidification generates. When such the cosmetics are used in the filling extrusion container, problems, such as non-uniform ejecting or clogging at the ejection portion, easily occurs at a time of ejection for use.

Therefore, the oily cosmetic having smooth usability, excellent makeup durability and temperature stability, and good ejection performance from the container has been desired, where the cosmetic is used in the filling extrusion container having excellent easiness of usage and portability. In addition, the cosmetic having no liquid leakage and dripping has been desired.

### Means for Solving the Problems

Present inventors carried out earnest works to solve the aforementioned problems and, as a result, they found out the followings to complete the present invention. That is, an oily cosmetic includes (a) 5 to 40 weight % dextrin palmitate having a substitution degree of acyl group is less than 2.0, and (b) 15 to 65 weight % volatile hydrocarbon oil. Further, the oily cosmetic includes 2 weight % or less solid oil having a melting point of 60° C. or higher. The oily cosmetic is used in a filling extrusion container, which is used by extruding a filling from a filling storage portion to a tip of ejection portion. The oily cosmetic has smooth usability, excellent makeup durability and temperature stability, and a good ejection performance. Further, in the oily cosmetic, liquid leakage and dripping are suppressed.

That is, the present invention provides the oily cosmetic including (a) 5 to 40 weight % dextrin palmitate having the substitution degree of acyl group of less than 2.0, and (b) 15 to 65 weight % volatile hydrocarbon oil. The oily cosmetic is used by filled and stored in a filling extrusion container, which is used by extruding a filling from a filling storage portion to a tip of ejection portion.

Further, the present invention provides the oily cosmetic including the content of a solid oil component of 2 weight % or less, where the solid oil component has a melting point of 60° C. or higher.

Furthermore, the present invention is the oily cosmetic in which, in the filling extrusion container, an inner diameter of the tip hole of the ejection portion is smaller than an inner diameter of the storage portion.

### Effect of the Invention

The oily cosmetic according to the present invention has smooth usability, excellent makeup durability and temperature stability, and good ejection performance from a container, and is suppressed from liquid leakage and dripping. The oily cosmetic can provide an oily cosmetic product having excellent simplicity in using method and portability.

### DETAILED DESCRIPTION OF PREFERRED EMBODIMENT

The exemplary embodiment according to the present invention will be described in detail as follows.

In addition, the following % indicates weight % and is shown hereinafter as %.

The present invention is the oily cosmetic including (a) 5 to 40 weight % dextrin palmitate having the substitution degree of acyl group of less than 2.0, and (b) 15 to 65 weight % volatile hydrocarbon oil. Further, the content of the solid oil having a melting point of 60° C. or higher is made to be 2 weight % or less in the oily cosmetic. The oily cosmetic is used in the filling extrusion container used by extruding the filling from the filling storage portion to the tip of ejection portion. The oily cosmetic has smooth usability, excellent makeup durability and temperature stability, and good ejection performance from the filling extrusion container, and is suppressed from liquid leakage and dripping.

As dextrin fatty acid esters for using for cosmetics, for example, dextrin laurate, dextrin myristate, dextrin palmitate, dextrin stearate, dextrin isopalmitate, dextrin isostearate, dextrin behenate, dextrin coconut oil fatty acid, dextrin oleate, dextrin palmitate/stearate mixed fatty acid, dextrin palmitate/2-ethylhexanoate, dextrin palmitate/octanoate, or the like can be used. In the present invention, (a) dextrin palmitate having the substitution degree of acyl group of less than 2.0 is used.

As (a) dextrin palmitate having the substitution degree of acyl group of less than 2.0, commercial products can be used. As an example of the commercial product, for example, Rheopear TL2 (made by Chiba Flour Milling Co., LTD) can be used.

The substitution degree of acyl group indicates an average mole number in which three hydroxyl groups in a monosaccharide unit are esterified with fatty acids. Further, the substitution degree of acyl group can be calculated by saponification values, or the like.

In the present invention, the content of the (a) dextrin palmitate having the substitution degree of acyl group of less than 2.0 is preferably 5 to 40% in the total amount of the oily cosmetic. As the lower limit of the content, 7% or more is more preferable, and 10% or more is specifically preferable. When the content is less than 5%, it is not preferable from the viewpoint of temperature stability and liquid dripping. Further, as the upper limit of the content, 30% or less is more preferable, and 20% or less is specifically preferable. When the content is more than 40%, it is not preferable from the viewpoint of ejection performance and usability feeling.

As the (b) volatile hydrocarbon oils used in the present invention, it is not particularly limited if the oils are usually used in cosmetics. In particular, volatile hydrocarbons oils such as light Isoparaffin, isododecane, or the like, can be used. One or more these volatile hydrocarbon oils can be suitably selected and used.

As the aforementioned volatile hydrocarbon oils, commercial products can be used. For example, as a light Isoparaffin, ISOPAR H (produced by Exxon Mobil corporation) and IP SOLVENT 1620 (produced by Idemitsu Kosan Co., Ltd) can be used and, as isododecane, Markasol R (produced by MARUZEN PETROCHEMICAL Co., LTD) and PERMETYL99A (produced by INEOS USA, LLC.) can be used.

In the present invention, the content of the aforementioned (b) volatile hydrocarbon oils is preferably 15 to 65%. As the lower limit of the content, 18% or more is more preferable, and 20% or more is specifically preferable. When the content is less than 15%, it is not preferable from the viewpoint of usability such as blurring and makeup durability. Further, as the upper limit of the content, 55% or less is more preferable, and 45% or less is specifically preferable. When the content is more than 65%, it is not preferable from the viewpoint of temperature stability and liquid dripping.

In the solid oily cosmetic of the present invention, in addition to the aforementioned essential components, other components conventionally used for cosmetics can be suitably blended within a range not disturbing the effects of the present invention. For example, liquid oil, solid oil, semi-solid oil, surfactants, film-forming agents, humectants, preservatives, antioxidants, ultraviolet absorbers, a drug, perfumes, and the like can be used.

As the liquid oil, which can be blended in the present invention, it is not particularly limited, if the liquid oil has a liquid form at a room temperature and is usually used in normal cosmetics. More specifically, hydrocarbon oils, such as, a liquid paraffin, squalane, and the like, ester oils, such as, diisostearyl malate, isopropyl myristate, cetyl 2-ethylhexanoate, isopropyl palmitate, ethylhexyl palmitate, glyceryl tricaprylate/caprate, glyceryl tri-2-ethylhexanoate, and the like, vegetable oils, such as, olive oil, castor oil, macadamia nut oil, jojoba oil, and the like, higher alcohol, such as, oleyl alcohol, octyl dodecanol, isostearyl alcohol, and the like, higher fatty acid, such as, isostearic acid, oleic acid, and the like, and silicone oils, such as, dimethyl polysiloxane, methylphenyl polysiloxane, decamethylcyclopentasiloxane, and the like, can be used. One or more kinds of these liquid oil components can be used.

As the solid oil and the semi-solid oil, which can be blended in the present invention, it is not particularly limited if these oils are usually used in normal cosmetics. More specifically, hydrocarbons, such as, petrolatum, paraffin wax, microcrystalline wax, polyethylene, and the like, vegetable fats and oils, such as, hydrogenated castor oil, hydrogenated jojoba oil, carnauba wax, rice wax, and the like, esters, such as, glyceryl tribehenate, cholesterol fatty acid esters, and the like, higher fatty acids, such as, stearic acid, behenic acid, and the like, higher alcohols, such as, stearyl alcohol, behenyl alcohol, and the like, and silicones, such as, alkyl-modified silicone, acrylic modified silicone, and the like can be used. In these solid oils and semi-solid oils, one or more kinds can be used for obtaining various usage feelings.

In the present invention, the content of the solid oils having a melting point of 60° C. or higher is preferably 2% or less. When the content is greater than 2%, the ejection performance from the filling extrusion container becomes poor, so that clogging at the ejection portion of the container or non-uniform ejection generate. As the result, the quality of the oily cosmetic is damaged so that it is not preferable.

As the powders, which can be blended in the present invention, it is not particularly limited if the powders are usually used in normal cosmetics. As examples of the powders, mica, kaolin, synthetic mica, silica, calcium carbonate, magnesium carbonate, aluminum oxide, red iron oxide, yellow iron oxide, black iron oxide, cobalt oxide, ultramarine, iron blue, titanium oxide, zinc oxide, pearl pigment (mica titanium, argentine, bismuth oxychloride, and the like), boron nitride, organic pigments (red No. 228, red No. 226, blue No. 404, and the like), polyethylene powder, polymethyl methacrylate, organopolysiloxane elastomers, and the like, can be used. The composite powders including two or more these powders can be used. Further, the powders, on which silicone compounds, metal soaps, amino acid compounds, fluorine compounds, or the like, are surface-treated by a public known method, can be also used. In these powders, for obtaining various usage feeling, or desired color tone when the cosmetic is used for makeup, one or more can be used.

The present invention is the oily cosmetic and the oily cosmetic products characterized to be used by filled and storied in the filling extrusion container, which is used by extruding the filling from the filling storage portion to the tip of ejection portion. The oily cosmetic and the oily cosmetic product have smooth usability and excellent makeup durability and temperature stability, and are suppressed from the liquid leakage and dripping. Further, the oily cosmetic has excellent ejection performance from the container. Since such a cosmetic is stored and filled in the container, it is preferable in the filling extrusion container that the inner diameter of the tip hole of the ejection portion is smaller than the inner diameter of the filling storage portion. Further, it is more preferable, in the filling extrusion container, that the inner diameter of the tip hole of the ejection portion is ½ or less than the inner diameter of the filling storage portion (when a shape of the filling storage portion is elliptical, or the like, the short diameter is taken). Furthermore, it is particularly preferable, in the filling extrusion container, that the inner diameter of the tip hole of the ejection portion is ⅕ or less than the inner diameter of the filling storage portion (when a shape of the filling storage portion is elliptical, or the like, the short diameter is taken). The shape of the ejection hole is not particularly limited and can suitably take circular, elliptical, rectangular, or the like. Further, in the case having ejection holes of two or more, the container can be preferably used. As the filling extrusion container having a smaller inner diameter of the tip hole of the ejection portion than the inner diameter of the storage portion, for example, a tube container made of lamination of a resin, such as low density polyethylene, and aluminum, lamination of two or more kinds of resin, lamination of a resin and aluminum, or the like, is discussed. This container is used by pushing, with a suitable outer force, the container body to extrude the filled cosmetic from the tip of ejection portion (refer to Japanese Patent Application Laid-Open No. 2011-251923). As another example, a filling extrusion container having a mechanism, in which a piston set at a rear end of the container moves forward and extrudes the filling to the ejection portion set at a tip of the filling extrusion container (refer to Japanese Patent Application Laid-Open No. 2000-262324), is discussed.

The oily cosmetic of the present invention can be obtained by blending the aforementioned components and treating according to a usual method. The oily cosmetic of the present invention can be used as cosmetics for skincare, makeup, and hair. Preferably, the oily cosmetic is suitable to use for eye shadow, eyebrow, eyeliner, mascara, blush, lipstick, foundation, concealer, a foundation for base, and the like. However, it is not limited in these usages.

### Embodiments

Hereinafter, the present invention will be specifically described by examples, but is not limited by these examples. The blending amount is weight %. Before description of the examples, effect test method used in the present invention will be described.

[Usability Test]

Each evaluation item of usage feeling (smoothness of usage), blurring (lack of blurring at a time of application), and makeup durability (good durability of makeup) is tested by expert panels of 10 persons, and an average point of 5 steps evaluation is acquired to determine based on the evaluation criteria below.

[Evaluation Criteria]
⊚; Average point is 4.0 or more.
◯; Average point is 3.0 or more and less than 4.0.
Δ; Average point is 2.0 or more and less than 3.0.
×; Average point is less than 2.0.

[Stability]

The cosmetic is poured and filled in a closed glass container, and allowed to stand for 24 hours at room temperature. Then, the container is allowed to stand for 1 week at 50° C. and the appearance is certified. The stability is determined by the following evaluation legends.
⊚; There is no change.
◯; There is a small amount of sweating on the surface.
Δ; There is a small amount of oil separation. (The separated oil is disappeared in 2 hours after returning a room temperature.)
×; There is a significant oil separation. (The separated oil is not disappeared over 2 hours after returning room temperature.)

[Ejection Performance]

The cosmetic is filled in a container having an extrusion mechanism by a piston, and allowed to stand for 24 hours at room temperature. Then, a time taken from the extrusion operation of the container to the ejection of the cosmetic, and an ejection state of such a time are measured, and the ejection performance is determined by the following evaluation legends.
⊚; The cosmetic is smoothly ejected at the same time as the extrusion operation of the container.
◯; After the extrusion operation of the container, the cosmetic is ejected at less than 1 second.
Δ; After the extrusion operation of the container, the cosmetic is ejected at 1 second or more and less than 3 seconds.
×; After the extrusion operation of the container, the cosmetic is ejected at 3 seconds or more, or a significant non-uniform ejection state.

[Liquid Leakage]

The cosmetic is filled in a container having an extrusion mechanism by a piston, and allowed to stand for 24 hours at room temperature. The container is allowed to vertically stand so as to put the tip of the ejection portion to be under for 1 week at 50° C. and presence or absence of liquid leakage and dripping to the tip of the ejection portion are certified. The determination is acquired by using the following evaluation legends.
◯; There is no liquid leakage at the tip of the ejection portion.
Δ; A little liquid leakage of the cosmetic is observed at the tip of the ejection portion.
×; A significant liquid leakage of the cosmetic is observed at the tip of the ejection portion.

In the above tests of [Ejection Performance] and [Liquid Leakage], as the container of the extrusion mechanism by the piston, the container described in Japanese Patent Application Laid-Open No. 2000-262324 (FIG. 1) is used. (Cosmetic storage portion volume; 1.8 mL, Inner diameter of the cosmetic storage portion; 8.6 mm, Inner diameter of the tip hole of the ejection portion ; 1.2 mm)

Oily cosmetics (an eyeliner) consisting of the components shown in Table 1 to Table 3 are prepared and made to be the evaluation samples. Each evaluation item of the evaluation samples is determined based on the above evaluation method. The results are shown in Table 1 to Table 3.

In addition, as raw materials of (Note 1) to (Note 12) shown in Table 1 to Table 3, the following commercial products are used, and the values of the blending amount indicates weight %.
(Note 1) Rheopear KL2 (Substitution degree of acyl group is 2.0 to 2.2); made by Chiba Flour Milling Co., LTD.
(Note 2) Rheopear TL2 (Substitution degree of acyl group is 1.5 to 1.7); made by Chiba Flour Milling Co., LTD.
(Note 3) Rheopear TT2; made by Chiba Flour Milling Co., LTD.
(Note 4) PERMETYL 99A; INEOS USA LLC.
(Note 5) Silicone KF-995; Shin-Etsu Chemical Co., Ltd.
(Note 6) BELSIL PMS MK POWDER SILICONE RESIN; Wacker Asahikasei Silicone Co., ltd.
(Note 7) SA/NAI-B10; Miyoshi Kasei, Inc.
(Note 8) SUNPMMA-S;SUNJIN CHEMICAL CO., LTD
(Note 9) Matsumoto Microsphere M503B; Matsumoto Yushi-Seiyaku Co., Ltd.
(Note 10) PERFORMALENE 500 (Melting potint of 95° C.); New Phase Technologies.
(Note 11) GIOVAREZ AC-5099M ; PHOENIX CHEMICAL, INC.
(Note 12); COLORONA MICA BLACK; Merck Ltd.

TABLE 1

| | | | Comparative | Comparative | Comparative |
|---|---|---|---|---|---|
| | | Example 1 | Example 1 | Example 2 | Example 3 |
| 1 | dextrin palmitate (Note 1) | — | 15 | — | — |
| 2 | dextrin palmitate (Note 2) | 15 | — | — | 15 |
| 3 | dextrin (palmitate/ethylhexanoate) (Note 3) | — | — | 15 | 0 |
| 4 | isotridecyl isononanoate | 5 | 5 | 5 | 5 |
| 5 | isododecane (Note 4) | 23.4 | 23.4 | 23.4 | — |
| 6 | decamethylcyclopentasiloxane (Note 5) | — | — | — | 23.4 |
| 7 | polymethylsilsesquioxane (Note 6) | 10 | 10 | 10 | 10 |
| 8 | silicone, amino acid processing black iron oxide (Note 7) | 33 | 33 | 33 | 33 |
| 9 | polymethylmethacrylate ball-like powder (Note 8) | 3 | 3 | 3 | 3 |
| 10 | talc | 10 | 10 | 10 | 10 |
| 11 | preservatives, antioxidants | 0.6 | 0.6 | 0.6 | 0.6 |

| | | | | |
|---|---|---|---|---|
| Total | 100 | 100 | 100 | 100 |
| usability | ⊚ | Δ | ⊚ | ⊚ |
| blurring | ⊚ | ⊚ | ◯ | X |
| makeup durability | ⊚ | ⊚ | ◯ | X |
| stability | ⊚ | ⊚ | X | X |
| ejection performance | ⊚ | X | ⊚ | ⊚ |
| liquid leakage | ◯ | ◯ | X | X |

(Preparation Method)

Example 1 and comparative examples 1 to 3 are prepared by the following method. The raw materials (1) to (7) are melted by heating, and then the raw materials (8) to (11) are added to be mixed and stirred. Then, after defoaming, the prepared cosmetic is filled in a container.

TABLE 2

| | | Comparative | | | | Comparative | Comparative |
|---|---|---|---|---|---|---|---|
| | | Example 4 | Example 2 | Example 3 | Example 4 | Example 5 | Example 6 |
| 1 | dextrin palmitate (Note 2) | 4 | 5 | 40 | 24 | 5 | 42 |
| 2 | ethylhexyl palmitate | 19.4 | 14.4 | — | — | — | — |
| 3 | isododecane (Note 4) | 13 | 20 | 49.5 | 59.4 | 65.4 | 32.4 |
| 4 | polymethylsilsesquioxane (Note 6) | 10 | 10 | 5 | 10 | 10 | 10 |
| 5 | silicone, amino acid processing black iron oxide (Note 7) | 30 | 30 | 5 | 5 | 14 | 10 |
| 6 | methylmethacrylate crosspolymer (Note9) | 23 | 20 | — | 1 | 5 | 5 |
| 7 | presetvatives, antioxidants | 0.6 | 0.6 | 0.6 | 0.6 | 0.6 | 0.6 |

| Total | 100 | 100 | 100 | 100 | 100 | 100 |
|---|---|---|---|---|---|---|
| usability | ◯ | ⊚ | ◯ | ⊚ | ⊚ | X |
| blurring | Δ | ◯ | ⊚ | ⊚ | X | ⊚ |
| makeup durability | Δ | ◯ | ⊚ | ⊚ | Δ | ⊚ |
| stability | X | ◯ | ⊚ | ⊚ | X | ⊚ |
| ejection performance | ⊚ | ⊚ | ◯ | ◯ | ⊚ | X |
| liquid leakage | Δ | ◯ | ◯ | ◯ | X | ◯ |

(Preparation Method)

Examples 2 to 4 and comparative examples 4 to 6 are prepared by the following method. The raw materials (1) to (4) are melted by heating, and then the raw materials (5) to (7) are added to be mixed and stirred. Then, after defoaming, the prepared cosmetic is filled in a container.

TABLE 3

| | | | | Comparative |
|---|---|---|---|---|
| | | Example 5 | Example 6 | Example 7 |
| 1 | dextrin palmitate (Note 2) | 7 | 5.5 | 4 |
| 2 | polyethylen wax (Note 10) | — | 1.5 | 3 |
| 3 | cetyl ethylhexanoate | 12.6 | 12.6 | 12.6 |
| 4 | isododecane(50%)/acrylate | 30 | 30 | 30 |
| | copolymer(50%) (Note 11) | | | |
| 5 | silica | 1 | 1 | 1 |
| 6 | black iron oxide | 30 | 30 | 30 |
| 7 | black iron oxide coated | 14 | 14 | 14 |
| | titanated mica (Note 12) | | | |
| 8 | polymethylmethacrylate ball- | 5 | 5 | 5 |
| | like powder (Note 8) | | | |
| 9 | preservatives, antioxidants | 0.4 | 0.4 | 0.4 |

| | | | |
|---|---|---|---|
| Total | 100 | 100 | 100 |
| usability | ⊚ | ◯ | X |
| blurring | ⊚ | ⊚ | ⊚ |
| makeup durability | ⊚ | ⊚ | ⊚ |
| stability | ⊚ | ⊚ | ⊚ |
| ejection performance | ⊚ | ◯ | X |
| liquid leakage | ◯ | ◯ | ◯ |

(Preparation Method)

Examples 5 to 6 and comparative example 7 are prepared by the following method. The raw materials (1) to (4) are melted by heating, and then the raw materials (5) to (9) are added to be mixed and stirred. Then, after defoaming, the prepared cosmetic is filled in a container.

As can be seen from Table 1 to Table 3, examples 1 to 6 are good in all evaluation items.

On the other hand, comparative example 1 using dextrin fatty acid ester, which is not claimed in the present invention, is inferior in ejection performance and usability. In the comparative example 2, stability and liquid leakage are inferior. In the both conflicting properties, such as, the ejection performance and the suppression of liquid leakage, good results can not be obtained.

Further, in the comparative example 3 using volatile oils, which is not the volatile hydrocarbon oil, enough gelation can not be obtained, so that the results are inferior, in not only the stability and the liquid leakage but also the blurring and the makeup durability.

In the comparative example 7, the ejection performance is inferior. After the extrusion operation of the container, 3 or more seconds are needed until ejection and the ejection state is non-uniform, so that the usability is inferior.

Examples 7 to 9 are described in the following as application examples.

### EXAMPLE 7

### Long Lasting Lip Color

| | |
|---|---|
| (Blending components) | (weight %) |

| | | |
|---|---|---|
| (1) | dextrin palmitate (Note 2) | 10.5 |
| (2) | glyceryl tri-2-ethylhexanoate | 5.0 |
| (3) | diisostearyl malate | 2.0 |
| (4) | natural vitamin E | 0.5 |
| (5) | isododecane | 45.0 |
| (6) | trimethylsiloxysilicate | 20.0 |
| (7) | mica titanium | 10.0 |
| (8) | Red No. 202 | 0.7 |
| (9) | Yellow No. 4 aluminum lake | 1.0 |
| (10) | Blue No. 1 aluminum lake | 0.1 |
| (11) | titanium oxide | 0.2 |
| (12) | Sericite | 5.0 |

(Production Method)

The blending components (1) to (6) are melted by heating, and the components (6) to (12) are added to be mixed and stirred. After defoaming, the produced cosmetic is directly filled in a tube-type container (a volume of the cosmetic storage portion is 12.5 ml, an inner diameter of the cosmetic storage portion is 18.5 mm, and an inner diameter of the tip hole of the ejection portion is 0.9 mm), and cooled, so that a long lasting lip color is obtained.

The above long lasing lip color in the example 7 has smooth usability, excellent makeup durability and temperature stability, and good ejection performance from the container. In addition, the long lasting lip color has no liquid leakage and dripping.

### EXAMPLE 8

### Eyeshadow

| | |
|---|---|
| (Blending components) | (weight %) |

| | | |
|---|---|---|
| (1) | dextrin palmitate (Note 2) | 20.0 |
| (2) | neopentyl glycol diethylhexanoate | 19.5 |
| (3) | isododecane | 25.0 |
| (4) | (trifluoropropyldimethylsiloxy/trimethylsiloxy) | 5.0 |
| | silsesquioxane (Note 13) | |
| (5) | dimethicone | 5.0 |
| (6) | natural vitamin E | 0.5 |
| (7) | mica | 5.0 |
| (8) | titanium mica | 15.0 |
| (9) | iron oxide | 5.0 |

(Production Method)

The blending components (1) to (5) are melted by heating, and the components (6) to (9) are added to be mixed and stirred. After defoaming, the produced cosmetic is directly filled in a tube-type container (the volume of the cosmetic storage portion is 12.5 ml, the inner diameter of the cosmetic storage portion is 18.5 mm, and the inner diameter of the tip hole of the ejection portion is 0.9 mm), and cooled, so that eyeshadow is obtained.
(Note 13) FR-10; Momentive Performance Materials Inc.

The above eyeshadow in the example 8 has smooth usability, excellent makeup durability and temperature stability, and good ejection performance from the container. In addition, the eyeshadow has no liquid leakage and dripping.

### EXAMPLE 9

### Lip Coat

| (Blending components) | | (weight %) |
|---|---|---|
| (1) | dextrin palmitate (Note 2) | 20.0 |
| (2) | Phenyltrimethicone | 6.0 |
| (3) | isododecane | 64.0 |
| (4) | (alkyl acrylate/dimethicone) copolymer (Note 14) | 10.0 |

(Production Method)

The blending components (1) to (4) are melted by heating, and mixed and stirred. Then, after defoaming, the mixed components are directly filled in a container described in Japanese Patent Application Laid-Open No. 2000-262324 (FIG. 1) (a cosmetic storage portion volume is 4.0 ml, an inner diameter of cosmetic storage portion is 11.8 mm, and an inner diameter of the tip hole of the ejection portion is 1.2 mm), and cooled, so that a lip coat is obtained.
(Note 14) KP 550; Shin-Etsu Chemical Co., Ltd.

The above lip coat in the example 9 has smooth usability, excellent makeup durability and temperature stability, and good ejection performance from the container. In addition, the lip coat has no liquid leakage and dripping.

What is claimed is:

1. An oily cosmetic comprising;
(a) dextrin palmitate of 5 to 40 weight %
(b) a volatile hydrocarbon oil of 15 to 65 weight %
wherein an acyl group substitution degree of the dextrin palmitate is less than 2.0,
wherein the oily cosmetic is used in a filling extrusion container, and
wherein the filling extrusion container is used by extruding a filling from a filling storage portion to a tip of ejection portion.

2. The oily cosmetic according to claim 1,
wherein a content of a solid oil having a melting point of 60° C. or higher is 2 weight % or less.

3. The oily cosmetic according to claim 1,
wherein, in the filling extrusion container, an inner diameter of the tip hole of the ejection portion is smaller than an inner diameter of the storage portion.

4. The oily cosmetic according to claim 2,
wherein, in the filling extrusion container, an inner diameter of the tip hole of the ejection portion is smaller than an inner diameter of the storage portion.
